(19) United States
(12) Patent Application Publication
(10) Pub. No.: US 2026/0167927 A1
(43) Pub. Date: Jun. 18, 2026

(54) GLOMUS CELL-LIKE CELLS AND PRODUCTION METHOD THEREOF

(71) Applicant: NATIONAL INSTITUTE OF ADVANCED INDUSTRIAL SCIENCE AND TECHNOLOGY, Tokyo (JP)

(72) Inventors: Yasuyuki KIDA, Tsukuba-shi, Ibaraki (JP); Yuka NIMIYA AKAGI, Tsukuba-shi, Ibaraki (JP)

(21) Appl. No.: 19/125,064

(22) PCT Filed: Sep. 4, 2023
(86) PCT No.: PCT/JP2023/032187

§ 371 Date: Apr. 28, 2025

(30) Foreign Application Priority Data
Oct. 31, 2022 (JP) 2022-174625

(51) Int. Cl. C12N 5/0793; C07K 14/435; C12N 5/10; C12Q 1/02

(52) U.S. Cl. CPC C12N 5/062; C07K 14/435; C12N 5/10; C12Q 1/025; C12N 2501/105; C12N 2501/11; C12N 2501/115; C12N 2503/02; C12N 2506/45

(57) ABSTRACT

Provided is a method for producing glomus cell-like cells, the method comprising: (a) a step for inducing differentiation of pluripotent stem cells into autonomic neural progenitor cells, and (b) a step for culturing the autonomic neural progenitor cells obtained by the step (a) in the presence of an FGF signaling pathway activator, an EGF signaling pathway activator, and an IGF-1 signaling pathway activator.

### TECHNICAL FIELD

The present invention relates to glomus cell-like cells and to production methods therefor.

### BACKGROUND ART

The carotid body (CB) is a peripheral chemoreceptor located at the bifurcation of the carotid artery. The CB comprises nerve-like glomus cells (also called type I cells) and glial-like supporting cells (also called type II cells), with glomus cells having a chemoreceptor function. When glomus cells sense hypoxia, hypercapnia, or acidosis, they release neurotransmitters such as ATP and dopamine, which stimulate the medulla oblongata via the glossopharyngeal nerve, resulting in increased respiratory rate and heart rate. Therefore, compounds that modulate the sensitivity and neurotransmitter-releasing activity of glomus cells are potential targets of drugs for respiratory failure treatment. Furthermore, in recent years, it has been found that glomus cells sense insulin and glucose and are involved in the maintenance of energy homeostasis, and they are also attracting attention as a new therapeutic target for diabetes mellitus.

Conventionally, non-human animal models have been used in CB research. However, testing a large number of candidate therapeutic drugs requires a considerable number of animals, which poses problems of being time consuming and incurring financial cost. Furthermore, in recent years, from an ethical standpoint, such as animal welfare and protection, there has been a strong demand for alternative test systems that do not use animals. In addition, there is a demand for a testing system closer to humans, considering differences between animal species. These situations demand the development of an in vitro CB model using human glomus cells.

On the other hand, CB-supporting cells have been reported as progenitor cells of glomus cells, and glomus cells can be produced from supporting cells in vitro (Patent Document 1 and Non-patent Document 1). However, since supporting cells need to be extracted from animals to produce glomus cells, there are difficulties in mass culturing human glomus cells in particular. To date, there is no reported method of producing glomus cells or supporting cells from human pluripotent stem cells.

### PRIOR ART

### Patent Document

Patent Document 1: International Publication No. 2009/016262

### Non-Patent Document

Non-patent Document 1: Pardal, R. et al., Cell, 2007; 131 (2): 364-377

### SUMMARY OF INVENTION

### Problem to be Solved by the Invention

The present invention aims to provide glomus cell-like cells that can reproduce the hypoxic response of CB in vitro, and a simple and efficient method to prepare them.

### Problem Solution

The inventors have previously developed a technique for efficiently inducing autonomic neurons or the progenitor cells thereof from human pluripotent stem cells (e.g., International Publication No. 2016/194522). By adding specific culture conditions to the above method, the present inventors succeeded in producing glomus cell-like cells that exhibit a hypoxic response from pluripotent stem cells.

In other words, according to one embodiment, the present invention provides a method for generating glomus cell-like cells, comprising (a) a step of inducing differentiation of pluripotent stem cells into autonomic neural progenitor cells, and (b) a step of culturing the autonomic neural progenitor cells obtained by the step (a) in the presence of an FGF signaling pathway activator, an EGF signaling pathway activator, and an IGF-1 signaling pathway activator.

It is preferred that the method further comprise (c) a step of introducing an exogenous nucleic acid encoding endothelial PAS domain-containing protein 1 (EPAS1) into the pluripotent stem cells, before the step (a).

The FGF signaling pathway activator is preferably bFGF.

The EGF signaling pathway activator is preferably EGF.

The IGF-1 signaling pathway activator is preferably IGF-1.

The pluripotent stem cells are preferably of human origin.

Furthermore, according to one embodiment, the present invention also provides glomus cell-like cells produced by the above method.

According to one embodiment, the present invention also provides a glomus cell-like cell comprising exogenous nucleic acid encoding EPAS1 and expressing tyrosine hydroxylase, potassium channel subfamily K member 3, and olfactory receptor 51E2.

The glomus cell-like cells preferably produce increased amounts of ATP, or dopamine or metabolites thereof, in hypoxic conditions.

Additionally, according to one embodiment, the present invention provides a method for screening a compound that regulates the activity of glomus cells, comprising (1) a step of contacting the above glomus cell-like cell with a candidate compound and (2) measuring ATP released from the glomus cell-like cell.

### Effects of the Invention

According to the method of the present invention, glomus cell-like cells can be easily produced with high efficiency. Also, the glomus cell-like cells of the present invention have excellent hypoxia responsiveness and are useful for developing in vitro CB models, searching for compounds that regulate CB activity, and developing therapeutic agents for CB-related diseases.

### BRIEF DESCRIPTION OF DRAWINGS

FIG. 1 is a schematic diagram showing the production schedule of glomus cell-like cells.

FIG. 2 shows brightfield microscopic images of embryoid bodies (EBs) on day 0 of induction, cells on day 13 of induction (autonomic neural progenitor cells), and cells on day 16 of induction (glomus cell-like cells).

FIG. 3 is a heat map showing the results of RNA-seq analysis of induced pluripotent stem (iPS) cells on day-3 of induction (in the figure, labeled “hiPSC”), cells on day 13 of induction (labeled “Progenitors”), and cells on day 16 of induction (labeled “Glomus cells”).

FIG. 4 is a graph showing the expression levels of the OR51E2 gene in EBs on day 0 of induction, cells on day 13 of induction (autonomic neural progenitor cells), and cells on day 18 of induction (glomus cell-like cells).

FIG. 5 is a graph showing the production of ATP by glomus cell-like cells in response to hypoxic stimulation.

FIG. 6 is a graph showing the production of epinephrine by glomus cell-like cells in response to hypoxic stimulation.

FIG. 7 is a graph showing the production of dopamine (DA) by glomus cell-like cells in response to hypoxic stimulation.

FIG. 8 is a graph showing the production of 3,4-dihydroxyphenylacetic acid (DOPAC) by glomus cell-like cells in response to hypoxic stimulation.

FIG. 9 is a graph showing the expression level of the TH gene in glomus cell-like cells in response to hypoxic stimulation.

FIG. 10 is a graph showing the expression level of the KCNK3 gene in glomus cell-like cells in response to hypoxic stimulation.

FIG. 11 is a graph showing the promoting effect of tetraethylammonium chloride (TEA) on ATP production by glomus-like cells in response to hypoxic stimulation.

FIG. 12 is a graph showing the inhibitory effect of nifedipine (Nif.) on ATP production in glomus-like cells in response to hypoxic stimulation.

FIG. 13 is a graph showing the inhibitory effect of lidocaine (Lid.) on ATP production by glomus-like cells in response to hypoxic stimulation.

FIG. 14 is a graph showing the expression level of the EPAS1 gene in induced pluripotent stem cells introduced with the exogenous EPAS1 gene.

FIG. 15 is a graph showing the expression level of the EPAS1 gene in cells prepared from induced pluripotent stem cells introduced with the exogenous EPAS1 gene on day 17 of induction.

FIG. 16 is a graph showing the expression levels of the TH gene in EPAS1-expressing iPS cells (undifferentiated, day-3 of induction, labeled “hiPSC” in the figure), autonomic neural progenitor cells derived from EPAS1-expressing iPS cells (day 13 of induction, labeled “Progenitors”), and EPAS1-overexpressing glomus cell-like cells (day 17 of induction, labeled “Glomus cells”).

FIG. 17 is a graph showing the expression levels of the KCNK3 gene in EPAS1-expressing iPS cells (undifferentiated, day-3 of induction), autonomic neural progenitor cells derived from EPAS1-expressing iPS cells (day 13 of induction) and EPAS1 overexpressing glomus cell-like cells (day 17 of induction).

FIG. 18 is a graph showing the expression levels of the OR51E2 gene in EPAS1-expressing iPS cells (undifferentiated, day-3 of induction), autonomic neural progenitor cells derived from EPAS1-expressing iPS cells (day 13 of induction) and EPAS1-overexpressing glomus cell-like cells (day 17 of induction).

FIG. 19 is a graph showing ATP production of EPAS1-overexpressing glomus cell-like cells under normoxic or hypoxic conditions.

### DESCRIPTION OF EMBODIMENTS

The present invention is described in detail below but is not limited to the embodiments described herein.

According to the first embodiment, the present invention is a method for producing glomus cell-like cells, comprising the steps of (a) inducing pluripotent stem cells to differentiate into autonomic neural progenitor cells and (b) culturing the autonomic neural progenitor cells obtained by the step (a) in the presence of an FGF signaling pathway activator, an EGF signaling pathway activator, and an IGF-1 signaling pathway activator.

In the method of the present embodiment, autonomic neural progenitor cells are induced from pluripotent stem cells. “Pluripotent stem cells” include, but are not limited to, embryonic stem (ES) cells, induced pluripotent stem (iPS) cells, embryonic germ (EG) cells, multipotent germline stem (mGS) cells, and Muse cells. In the method of the present embodiment, any pluripotent stem cells may be used, but ES cells or iPS cells are preferred, and iPS cells are even more preferred.

The pluripotent stem cells in the present embodiment may be derived from any vertebrate, preferably from mammals, such as mice, rats, rabbits, sheep, goats, pigs, cows, monkeys, humans, and especially from humans.

Methods for preparing pluripotent stem cells are well established (e.g., for iPS cells, see Cell, 2007; 131 (5): 861-872, doi: 10.1016/j.cell.2007.11.019), and pluripotent stem cells can be prepared from any tissue or cell according to methods known in the field. Alternatively, established iPS or ES cell lines may be obtained from, for example, the Kyoto University iPS Cell Research Foundation (CiRA_F), the RIKEN Bioresource Research Center (RIKEN BRC), or the American Type Culture Collection (ATCC).

“Autonomic neural progenitor cells” in the present embodiment refer to neural crest cells or cells that are more differentiated into autonomic neurons, and that can differentiate into sympathetic and parasympathetic neurons. In the present embodiment, autonomic neural progenitor cells can be defined, for example, based on the expression of SOX10 (neural crest cell marker gene) and PHOX2B (autonomic neuron marker gene).

Methods for inducing pluripotent stem cells into autonomic neural progenitor cells are well established (e.g., WO 2020/040286, WO 2016/194522), and autonomic neural progenitor cells can be prepared according to methods known in the field. Specifically, for example, autonomic neural progenitor cells can be prepared by culturing pluripotent stem cells under conditions in which a BMP signaling pathway inhibitor such as dorsomorphin, a TGF signaling pathway inhibitor such as SB431542, a Wnt signaling pathway activator such as CHIR99021, or an FGF signaling pathway activator such as basic fibroblast growth factor (bFGF), are appropriately added to a basic medium such as DMEM/HAM's F-12 medium, human stem cell (hES) medium, N2 medium, or a mixture thereof. In this case, the pluripotent stem cells are preferably cultured for 2 to 3 days in a medium containing a Rho kinase (ROCK) inhibitor, such as Y-27632, prior to the above differentiation induction.

Autonomic neural progenitor cells can be stably subcultured in a medium supplemented with EGF or bFGF until they are induced to differentiate into glomus cell-like cells while maintaining their differentiation potential (Fukuta, M., et al., PLOS ONE, 9 (12): e112291, 2014).

Next, autonomic neural progenitor cells are cultured in the presence of an FGF signaling pathway activator, an EGF signaling pathway activator, and an IGF-1 signaling pathway activator. This allows autonomic neural progenitor cells to be induced into glomus cell-like cells.

The “FGF signaling pathway activator” in the present embodiment may be any known compound that activates the FGF (fibroblast growth factor) receptor and a downstream signaling pathway thereof, including, for example, acidic fibroblast growth factor (FGF1, aFGF), which belongs to the FGF1 family, basic fibroblast growth factor (FGF2, bFGF), FGF-G3™, which is a recombinant form of FGF2, fibroblast growth factor chimera (FGFC), but is not limited to these. The FGF signaling pathway activator in the present embodiment is preferably bFGF, and the concentration of bFGF in the medium can preferably be 5 to 50 ng/mL.

The “EGF signaling pathway activator” in the present embodiment may be any known compound that activates the EGF receptor and a downstream signaling pathway thereof, including, for example, EGF, TGF-β, amphiregulin (AREG), heparin-binding EGF-like growth factor (HB-EGF), betacellulin (BTC), epigene (EPG), and epiregulin (EPR), but is not limited to these. The EGF signaling pathway activator in the present embodiment is preferably EGF, and the concentration of EGF in the medium can preferably be 5 to 50 ng/ml.

The “IGF-1 signaling pathway activator” in the present embodiment may be any known compound that activates the IGF-1 (insulin-like growth factor 1) receptor and a downstream signaling pathway thereof, including, for example, IGF-1, IGF-2, and insulin, but is not limited to these. The IGF-1 signaling pathway activator in the present embodiment is preferably IGF-1, and the concentration of IGF-1 in the medium can preferably be 5 to 50 ng/ml.

The medium used here may be, for example, DMEM/HAM's F-12 medium as the base medium, with fetal bovine serum (FBS), penicillin-streptomycin, nonessential amino acid solution, N2 supplement, B-27 supplement, etc., added as needed. Any equivalent substitute may be used in place of FBS, and such substitutes include, but are not limited to, KnockOut Serum Replacement (KSR) (Thermo Fisher Scientific: 10828028), StemSure™ Serum Replacement (SSR) (FUJIFILM Wako Pure Chemical Corporation: 191-18375), XF212 XerumFree (TNC BIO BV:XF212-0100-1s), etc. FBS or substitute thereof may be added, preferably at a concentration of 10% to 20%. For example, autonomic neural progenitor cells may be seeded at concentrations ranging from 1×10⁶ to 1×10⁸ cells/mL. The culture period in the presence of FGF signaling pathway activators, EGF signaling pathway activators, and IGF-1 signaling pathway activators may be 3 to 50 days, for example, preferably 3 to 7 days. For example, autonomic neural progenitor cells derived from mammals are preferably cultured at 37° C. and 5% CO₂.

The method of the present embodiment preferably introduces exogenous nucleic acids that encode factors associated with the development and/or function of glomus cells (hereinafter referred to as “glomus cell-associated factors”). This makes it possible to produce glomus cell-like cells with improved function. Glomus cell-associated factors include, but are not limited to, endothelial PAS domain-containing protein 1 (EPAS1, also known as HIF-2α), brain-derived neurotrophic factor (BDNF), paired-like homeobox 2b (PHOX2B), SRY-BOX transcription factor 4 (SOX4), SRY-BOX transcription factor 11 (SOX11), and hypoxia-inducible factor 1A (HIF1A). In the method of the present embodiment, it is particularly preferable to introduce an exogenous nucleic acid encoding EPAS1.

The exogenous nucleic acid encoding glomus cell-associated factors may be introduced into the cell at any time. For example, the exogenous nucleic acid encoding glomus cell-associated factors may be introduced into pluripotent stem cells prior to induction of differentiation, into autonomic neural progenitor cells obtained by inducing differentiation, or into autonomic neural progenitor cells that are differentiating into glomus cells. In the method of the present embodiment, exogenous nucleic acid encoding glomus cell-associated factors are preferably introduced into pluripotent stem cells prior to induction of differentiation.

In the present embodiment, the exogenous nucleic acid encoding the glomus cell-associated factor can be derived from any vertebrate, preferably from mammals, such as mice, rats, sheep, goats, pigs, cows, monkeys, and humans, and especially from humans. Genes encoding glomus cell-associated factors have already been cloned, and their nucleotide sequence information can be obtained from a designated database. For example, NM_001430.5 for the human EPAS1 gene, NM_001143805.1 for the human BDNF gene, NM_003924.4 for the human PHOX2B gene, NM 003107.3 for the human SOX4 gene, NM_003108.4 for the human SOX11 gene, NM 00124308.2 for the human HIF1A gene (all NCBI Ref Seq IDs) are available.

In the present embodiment, glomus cell-associated factors may include variants and homologs with equivalent activity. In other words, the glomus cell-associated factor in the present embodiment can include proteins comprising an amino acid sequence that has more than 80%, preferably more than 90%, and more preferably about 95% or more identity with the amino acid sequences in the database, provided that the physiological activity is maintained. The identity of the amino acid sequence can be calculated using sequence analysis software or programs customary in the field (FASTA, BLAST, etc.). The glomus cell-associated factor in the present embodiment may include proteins consisting of amino acid sequences in which one to a few amino acids are substituted, deleted, inserted, and/or added in the amino acid sequence registered in the database, provided that the physiological activity is maintained. Here, “one to a few” means, for example, 1 to 30, preferably 1 to 10, and especially preferably 1 to 5.

The exogenous nucleic acids encoding glomus cell-associated factors can be introduced into cells by methods that are well known in the art, for example, by cloning the nucleic acids into an expression vector and introducing it into cells. Examples of expression vectors include, but are not limited to, viral vectors such as retrovirus, lentivirus, adenovirus, Sendai virus, and plasmid vectors such as pCMV.

In the method of the present embodiment, the expression vector can be introduced into cells by methods well-known in the art, depending on its type. If a non-viral vector is used, it can be introduced, for example, by lipofection, electroporation, or microinjection. If a viral vector is used, it can be introduced by infecting cells at the appropriate titer or multiplicity of infection (MOI).

According to the method of the present embodiment, it is possible to produce glomus cell-like cells.

According to the second embodiment, the present invention is a glomus cell-like cell produced by the above method.

Here, the term “glomus cell-like cells” refers to cells that express tyrosine hydroxylase (TH), potassium channel subfamily K member 3 (KCNK3), and olfactory receptor 51E2 (OR51E2), marker genes that are expressed in glomus cells, and that can mimic the hypoxic response of glomus cells. Therefore, it is preferable that the glomus cell-like cells of the present embodiment express EPAS1 in addition to the above markers. It is more preferable for the present embodiment of glomus cell-like cells to further express ubiquitin C-terminal hydrolase L1 (UCHL1), heme oxygenase (HO-2), Maxi-K channel (Maxi-K), class III β tubulin (TUBB3), hypoxia-inducible factor 1A (HIF1A), and/or dopamine receptor (DRD2).

In other words, according to a third embodiment, the invention is a glomus cell-like cell that contains an exogenous nucleic acid encoding endothelial PAS domain-containing protein 1 (EPAS1), and expresses tyrosine hydroxylase (TH), potassium channel subfamily K member 3 (KCNK3), and olfactory receptor 51E2 (OR51E2). The “exogenous nucleic acid” in the present embodiment is as defined in the first embodiment.

The glomus cell-like cells of the present embodiment preferably overexpress EPAS1. “Overexpression of EPAS1” refers to the state in which EPAS1 is expressed more than the expression level of EPAS1 in glomus cell-like cells prepared without introducing exogenous nucleic acid encoding EPAS1.

The expression of markers and EPAS1 can be analyzed by known methods such as RT-PCR, Western blotting, and flow cytometry.

In addition to the expression of markers and EPAS1, glomus cell-like cells in the present embodiment may be defined based on hypoxia responsiveness, for example, increased production of ATP or dopamine or metabolites thereof under hypoxic conditions. Metabolites of dopamine include, but are not limited to, tyrosine, L-DOPA, 3,4-dihydroxyphenylacetic acid (DOPAC), epinephrine, and norepinephrine, etc. ATP or dopamine or metabolites thereof may be measured by methods well known in the art, such as liquid chromatography-mass spectrometry (LC-MS/MS) or ELISA.

Here, “hypoxic condition” refers to when the oxygen supply to the cells is below physiological levels. A hypoxic condition can be induced by culturing cells in an atmosphere with an oxygen concentration of 10%, 5%, 3%, 2% or less. On the other hand, “normoxic condition” refers to the state when the oxygen supply to the cells is at the physiological level, and experimentally means culturing in an atmosphere with the oxygen concentration of atmospheric air (approximately 21%).

The second and third embodiments of glomus cell-like cells can reproduce the hypoxic response of glomus cells in vitro, and are useful for creating in vitro CB models.

According to a fourth embodiment, the present invention is a method for screening compounds that regulate the activity of glomus cells, comprising the steps of (1) contacting the abovementioned glomus cell-like cell with a candidate compound and (2) measuring the ATP released from the glomus cell-like cell.

The “candidate compound” in the present embodiment may be a small molecule compound, a nucleic acid, a protein, a peptide, an antibody, a lipid, or a mixture thereof (e.g., an extract from a cell or tissue, a cell or tissue culture supernatant, etc.). These candidate compounds may be new or be publicly known. In the method of the present embodiment, commercially available compound libraries may be used, for example, the Standard Compound Library (RIKEN NPDepo), Osaka University Original Compound Library (Osaka University), InhibitorSelect™ Libraries (Merck), and SCREEN-WELL™ Compound Library (ENZO LIFE Sciences, INC.) are preferred compound libraries.

To contact a candidate compound with the glomus cell-like cells, the glomus cell-like cells can be cultured for a certain time in a medium supplemented with the candidate compound. The concentration of the candidate compound to be added depends on the type of candidate compound. For example, the concentration can be selected from 1 μM to 100 mM for a small molecule compound. For example, the culture period may be from 1 second to 72 hours.

Next, the ATP released from the glomus cell-like cells is then measured. ATP may be measured by well-known methods, such as liquid chromatography-mass spectrometry (LC-MS/MS) or ELISA. Kits for ATP measurement are commercially available, and such commercial products can be used in this method. For example, ATP Determination Kit (Thermo Fisher Scientific) is a preferred commercial product.

To determine whether the addition of a candidate compound has altered ATP production, a culture without the addition of the candidate compound may be analyzed in parallel for comparison, or the results of previous analyses of cultures without the candidate compound may be compared. In the method of the present embodiment, if the release of ATP from glomus cell-like cells in the medium with the candidate compound is significantly increased or decreased compared to glomus cell-like cells in a medium without the candidate compound, the candidate compound can be determined as a promising compound for regulating glomus cell activity. Compounds promoting glomus cell activity may be useful, for example, in treating respiratory failure, hypotension, hypoglycemia, and dysfunction due to infection. Compounds that inhibit glomus cell activity may be useful, for example, in treating diabetes, hypertension, altitude sickness, hyperactivity due to infection, hyperventilation, carotid body tumors (also known as paragangliomas, glomus tumors), etc.

### EXAMPLES

Hereinafter, the present invention will be further described with reference to Examples. However, the present invention is not in any way limited thereto.

<1. Induction of Differentiation from iPS Cells to Glomus Cell-Like Cells>

### (1-1) Reagents

The reagent information used in this example (reagent name, product number, manufacturer, abbreviation, etc.) is as follows:
mTeSR1-cGMP (STEMCELL Technologies: ST-85850G) (hereinafter referred to as “mTeSR1”)
DMEM/Ham's F-12 (FUJIFILM Wako Pure Chemical Corporation: 048-29785)
DMEM (high-glucose) (FUJIFILM Wako Pure Chemical Corporation: 043-30085)
Opti-MEM (Thermo Fisher Scientific: 31985062)
STEM-CELLBANKER™ GMP grade (ZNQ: CB045)
Fetal Bovine Serum (Biowest, Nuaille, France) (hereinafter referred to as “FBS”)
Knockout Serum Replacement (Thermo Fisher Scientific: 10828-028) (hereinafter referred to as “KSR”)
N2 supplement with transferrin (Apo) (FUJIFILM Wako Pure Chemical Corporation: 141-09041) supplement (hereinafter referred to as “N2”)
MEM nonessential amino acids solution (FUJIFILM Wako Pure Chemical Corporation: 139-15651) (hereinafter referred to as “NEAA”)
B-27 supplement (Thermo Fisher Scientific: 17504044) (hereinafter referred to as “B27”)
Monothioglycerol solution (FUJIFILM Wako Pure Chemical Corporation: 195-15791)
Penicillin-streptomycin solution
(FUJIFILM Wako Pure Chemical Corporation: 168-23191) (hereinafter referred to as “P/S”)
Ethanol (99.5) (FUJIFILM Wako Pure Chemical Corporation: 057-00456)
Y-27632 (FUJIFILM Wako Pure Chemical Corporation: 036-24023)
Forskolin (FUJIFILM Wako Pure Chemical Corporation: 067-02191) (hereinafter referred to as “FSK”)
SB431542 hydrate (Sigma-Aldrich: (S4317-5 MG) (hereinafter referred to as “SB”)
CHIR99021 (Cayman Chemical Company: 13122) (hereinafter referred to as “CHIR”)
IWR-1 (Sigma-Aldrich: I0161-5 MG)
SANT1 (Sigma-Aldrich: S4572-5 MG)
Bone morphogenetic factor 4 (truncated) human recombinant (FUJIFILM Wako Pure Chemical Corporation: 022-17071) (hereinafter referred to as “BMP4”)
Fibroblast growth factor (basic FGF), human, recombinant (FUJIFILM Wako Pure Chemical Corporation: 064-04541) (hereinafter referred to as “bFGF”)
Epidermal Growth Factor (EGF) (FUJIFILM Wako Pure Chemical Corporation: 059-07873)
Insulin-like growth h factor-1 (IGF-1) (FUJIFILM Wako Pure Chemical Corporation: 096-05741)
iMatrix-511 (Nippi: 892012)
Lipidure (NOF Corporation: CM5206)
Accutase (Thermo Fisher Scientific: A11105-01)
Tryp LE express (Thermo Fischer Scientific: 12604-013)
Ultrapure distilled water (Thermo Fisher Scientific: 10977-015) (hereinafter referred to as “DW”)
D-PBS (−) (FUJIFILM Wako Pure Chemical Corporation: 045-29795) (hereinafter referred to as “PBS”)
Tris buffer powder, pH7.4 (Takara Bio: T9153)
Hydrochloric acid (FUJIFILM Wako Pure Chemical Corporation: 080-01066)
Albumin, from Bovine Serum (FUJIFILM Wako Pure Chemical Corporation: 017-23294) (hereinafter referred to as “BSA”)
Dimethyl sulfoxide (FUJIFILM Wako Pure Chemical Corporation: 046-21981) (hereinafter referred to as “DMSO”)

### (1-2) Stock Solution

FSK: Prepared to 10 mM using DMSO
DM: Prepared to 1 mM using DMSO
SB: Prepared to 10 mM using DMSO
CHIR: Prepared to 3 mM using DMSO
IWR-1: Prepared to 10 mM using DMSO
SANT1: Prepared to 250 μM using DMSO.
BMP4: Prepared to 100 μg/mL using 4 mM hydrochloric acid solution containing 0.1% BSA.
bFGF: Prepared to 500 μg/mL with 1 mM Tris buffer (pH 7.4) and then prepared to 10 μg/mL with DMEM
EGF and IGF-1: Prepared to 20 μg/mL using PBS
Ascorbic acid: Prepared to 50 mg/mL using PBS.
Y-27632: Prepared to 10 mM using Opti-MEM
Lipidure: Prepared to 0.5% with ethanol (99.5)

### (1-3) Culture Medium

Human stem cell medium (hESM): DMEM/Ham's F-12 supplemented with 20% KSR, 1% NEAA, 1% monothioglycerol solution, and 1% P/S
N2 medium: DMEM/Ham's F-12 supplemented with 1% N2, 1% NEAA, and 1% P/S
Glomus cell differentiation medium (GDM): DMEM/Ham's F-12 supplemented with 15% FBS, 1% N2, 2% B27, 20 ng/ml of bFGF, 20 ng/ml of EGF, 20 ng/ml of IGF-1 and 1% P/S
   (1-4) Induction of Differentiation of iPS Cells into Glomus Cell-Like Cells
   Formation of embryoid bodies (Day-3):

Human iPS cells (strain 201B7) were obtained from RIKEN BRC.

A 6-well plate was coated with Lipidure and washed with PBS. Human iPS cells (strain 201B7) were seeded at a concentration of 1×10⁶ cells/well with mTeSR1 supplemented with 10 μM of Y-27632 and cultured on an orbital shaker (WakenBtech: WB-101S RC) (95 rpm). Twenty-four hours and 48 hours after seeding, mTeSR1 with 10 μM of Y-27632 was added at 2 mL/well. The cells were cultured for 3 days until EBs formed.

### Differentiation Step 1 (Day 0):

The medium was replaced with hESM supplemented with 2 μM of DM, 10 μM of SB, and 10 ng/ml of bFGF (4 mL/well) and cultured on an orbital shaker for 2 days.

### Differentiation Step 2 (Day 2):

The medium was replaced with hESM supplemented with 3 μM of CHIR, 20 μM of SB, and 10 ng/ml of bFGF (6 mL/well) and cultured on orbital shakers for 3 days.

### Differentiation Step 3 (Day 5):

The medium was replaced with hESM: N2 (3:1) mixed medium supplemented with 3 μM of CHIR and 10 ng/ml of bFGF (4 mL/well), and cultured on an orbital shaker for 2 days.

Differentiation step 4 (Day 7):

The medium was replaced with hESM:N2 (1:1) mixed medium supplemented with 10 μM of IWR-1, 250 nM of SANT1, 25 ng/ml of BMP4, and 10 ng/ml of bFGF (3 mL/well), and cultured on an orbital shaker for 2 days.

### Differentiation Step 5 (Day 9):

The medium was replaced with hESM:N2 (1:3) mixed medium supplemented with 10 μM of IWR-1, 250 nM of SANT1, 25 ng/mL of BMP4, and 10 ng/ml of bFGF (3 mL/well), and cultured on an orbital shaker for 3 days. The medium was then replaced with fresh medium and cultured for another 24 hours.

### Differentiation Step 6 (Day 13):

The medium was replaced with GDM (3 mL/well) and cultured on orbital shakers for 3 to 4 days.

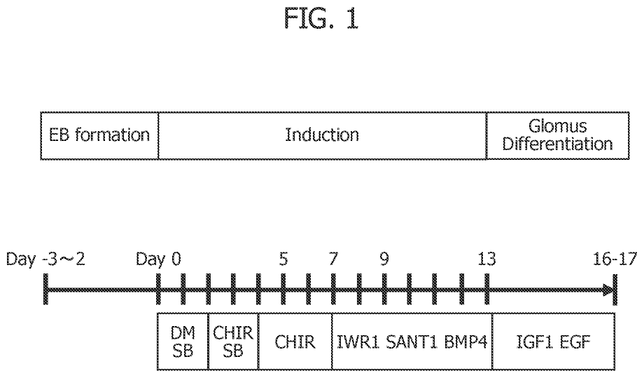

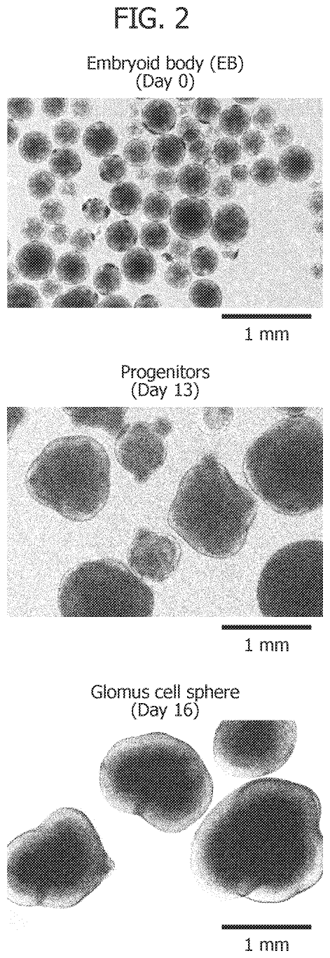

An outline of the schedule for producing glomus cell-like cells is shown in FIG. 1. Microscopic images (bright field) of EBs on day 0 of induction, cells on day 13 of induction (autonomic neural progenitor cells), and cells on day 16 of induction (glomus cell-like cells) are shown in FIG. 2.

### <2. Gene Expression in Glomus Cell-Like Cells>

Total RNA was extracted from iPS cells (undifferentiated, day 3 of induction), autonomic neural progenitor cells (day 13 of induction), and glomus cell-like cells (day 16 of induction) using NucleoSpin RNA. The library for RNA-seq was prepared using TruSeq-stranded mRNA (Illumina). Sequences were determined using NovaSeq 6000 (Illumina). Mapping and quantification were performed using STAR (2.7.1a) and RSEM (1.3.1). As the reference genome, hg38 was used, and Ensembl GRCh38 was used as the gene annotation. Differentially expressed genes were analyzed using the edgeR package (version 3.24.3) of the statistical analysis software R (version 3.5.1).

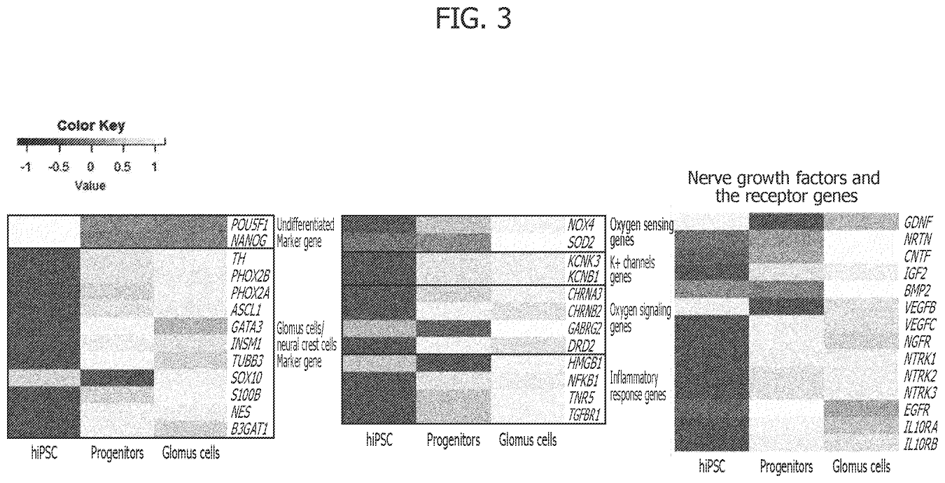

The results are shown in FIG. 3. In glomus cell-like cells, the expressions of TH, which is a glomus cell marker, genes related to hypoxic and immune responses, and nerve growth factors and receptor genes which are known to be expressed in mouse and rat glomus cells, were found to be increased.

Next, the expression of OR51E2 in iPS cells, autonomic neural progenitor cells, and glomus cell-like cells, which were under normoxic conditions, was then analyzed by quantitative PCR (qPCR). qPCR was performed using the same procedure as (3-3) below.

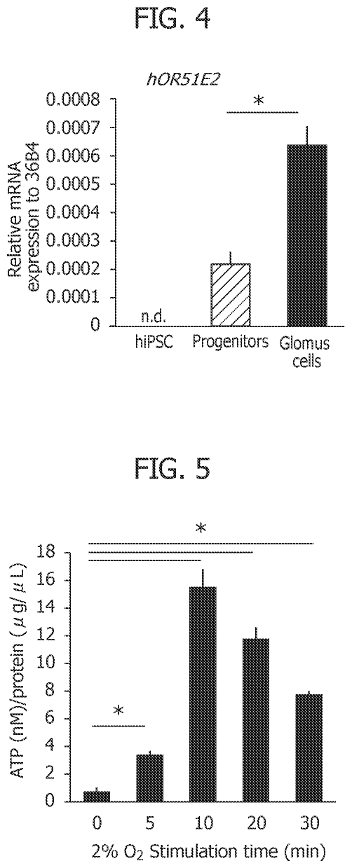

The results are shown in FIG. 4. In the figure, “hiPSCs” refers to iPS cells (undifferentiated, day 13 of induction), “progenitors” refers to autonomic neural progenitor cells (day 13 of induction), and “glomus cells” refers to glomus cell-like cells (day 16 of induction). It was confirmed that OR51E2 expression increased with differentiation (*P<0.05, n=3, Student's t-test).

### <3. Hypoxic Response of Glomus Cell-Like Cells>

### (3-1) Production of ATP

Glomus cell-like cells on days 16 to 19 of induction were used. With Krebs-Ringer buffer (KRB: 120 mM of NaCl, 5 mM of KCI, 25 mM of NaHCO₃, 2.5 mM of CaCI2, 1.1 mM of MgCl₂, 0.1% bovine serum albumin, 2.8 mM of glucose, pH 7.2), glomus cell-like cells were washed twice. After adding KRB, the cells were incubated in a hypoxic CO₂ incubator (2% O₂) for 10 to 30 minutes. The supernatant was collected, and then ATP was measured using the ATP Determination Kit (Thermo Fisher Scientific: A22066). Cells were lysed in RIPA buffer (FUJIFILM Wako Pure Chemical Corporation: 182-02451), and the total protein content was determined using a Pierce™ BCA Protein Assay Kit (Thermo Fisher Scientific: 23225). ATP measurement results were corrected according to the total protein content.

The results are shown in FIG. 5. It was confirmed that ATP production from glomus cell-like cells increased in response to hypoxic stimulation (*P<0.05, n=3, Student's t-test).

### (3-2) Production of Catecholamines

Glomus cell-like cells on days 16 to 19 of induction were used. Hypoxic stimulation was performed by the same procedure as (3-1) above. KRB was removed, and the cells were suspended in a buffer (10 mM phosphate buffer, pH 5.0) used as the mobile phase of high-performance liquid chromatography (HPLC). Then, cells were disrupted by sonication (3 sets of 10 sec), and were centrifuged at 12,000×g for 5 min at 4° C., and the supernatant was subjected to a protein concentrator to remove proteins from the solution. The solution obtained was subjected to HPLC to measure epinephrine, dopamine (DA), and DOPAC.

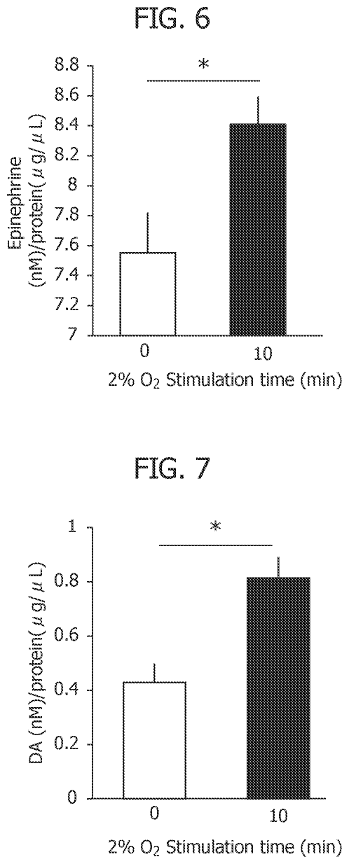

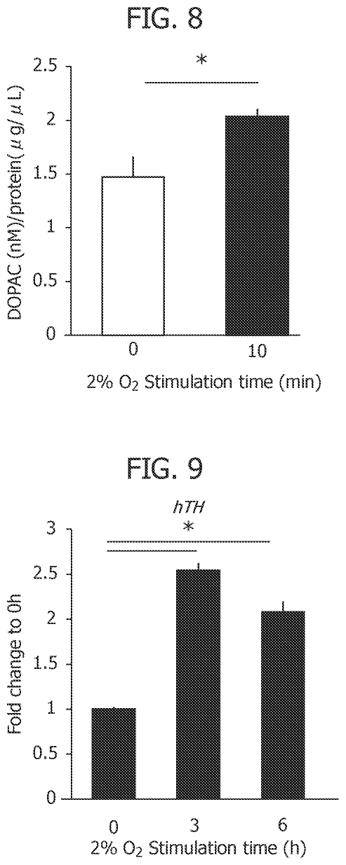

The measurement results for epinephrine are shown in FIG. 6, and the measurement results for DA are shown in FIG. 7, and the measurement results for DOPAC are shown in FIG. 8. It was confirmed that the production amounts of epinephrine, DA, and DOPAC increased in response to hypoxic stimulation (*P<0.05, n=3, Student's t-test).

### (3-3) Gene Expression

Gene expression in glomus cell-like cells was analyzed by qPCR. Hypoxia stimulation was performed by the same procedure as described above (3-1). Total RNA was extracted using Nucleo Spin RNA (Takara Bio, U0955B). ReverTra Ace™ qPCR RT Master Mix with gDNA Remover (Toyobo, FSQ-301) was used to prepare cDNA. qPCR was performed using a THUNDERBIRD™ SYBR™ qPCR Mix (Toyobo, QPS-201). The housekeeping gene 36B4 was used as an internal control. Reactions and analyses were performed in triplicate.

TABLE 1
Primer sets used for qPCR

| Primer | Sequence (5′→3′) | SEQ NO. |
|---|---|---|
| 36B4-F | AGATGCAGCAGATCCGCA | 1 |
| 36B4-R | GTTCTTGCCCATCAGCACC | 2 |
| hTH-F | GCGCAGGAAGCTGATTGC | 3 |
| hTH-R | CAATCTCCTCGGCGGTGTAC | 4 |
| hKCNK3-F | CTACGAGCACTGGACCTTCTT | 5 |
| hKCNK3-R | CGTAAGGATGTAGACGAAGCTGA | 6 |
| hOR51E2-F | CCGAACTGCTGTATGGGCTC | 7 |
| hOR51E2-R | GCATGTGTGAAGTTGCAGGAA | 8 |
| hEPAS1-F | GTCTGCAAAGGGTTTTGGGG | 9 |
| hEPAS1-R | TGTGAGGTGCTGCCACCAG | 10 |

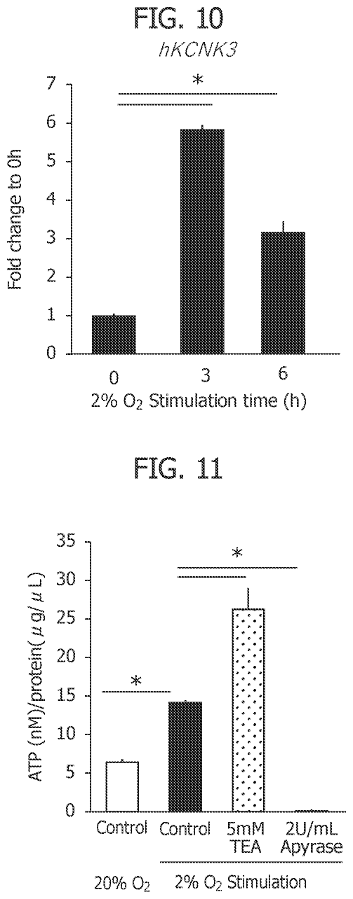

The expression levels of TH and KCNK3 are shown in FIG. 9 and FIG. 10, respectively. It was confirmed that the expression levels of both TH and KCNK3 increased with hypoxic stimulation (*P<0.05, n=3, Student's t-test).

### (3-4) Changes in Hypoxic Response as a Result of Ion Channel Inhibition

Using KRB containing the K⁺ channel inhibitor tetraethylammonium chloride (FUJIFILM Wako Pure Chemical Corporation: 206-04501) (final concentration: 5 mM), the Ca²⁺ channel inhibitor nifedipine (FUJIFILM Wako Pure Chemical Corporation: 141-05783) (final concentration: 5 μM), or the Na⁺ channel inhibitor lidocaine (FUJIFILM Wako Pure Chemical Corporation: 120-02691) (final concentration: 1 μM), cells were subjected to hypoxic stimulation using the procedure described in (3-1) above, and the amount of ATP production was analyzed. KRB without ion channel inhibitors was used as a control to analyze ATP production similarly.

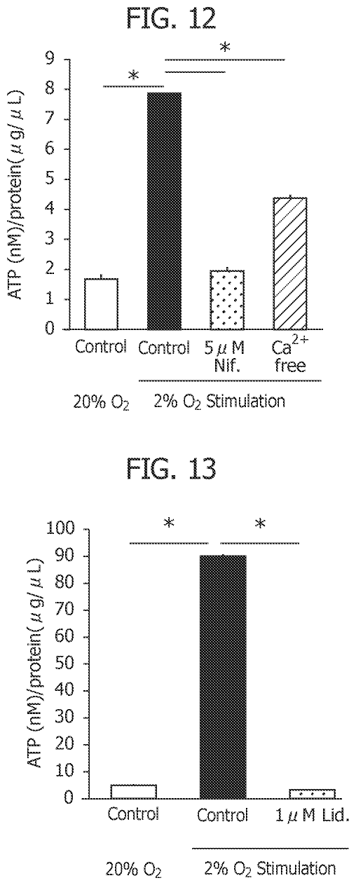

Results are shown in FIGS. 11 to 13 (*P<0.05, n=3, Student's t-test). Inhibition of K⁺ channels increased ATP production in response to hypoxic stimulation (FIG. 11). When an ATPase, apyrase (Sigma-Aldrich: A6132-200UN) (final concentration: 2 U/mL), was added, no ATP was detected (FIG. 11). In contrast, inhibition of Ca²⁺ or Na⁺ channels reduced ATP production in response to hypoxic stimuli (FIGS. 12 and 13). When KRB without Ca²⁺ was used, ATP production in response to hypoxic stimulation was also reduced (FIG. 12). These results confirm that glomus cell-like cells prepared by the procedure (1-4) above exhibit a hypoxic response by the same molecular mechanism as glomus cells.

<4. Generation of iPS Cells with Forced Expression of EPAS1>

VectorBuilder was commissioned to synthesize a plasmid containing the EPAS1 gene (NCBI RefSeq ID: NM_001430.5) (pLV-hEPAS1, vector ID: VB11202-1497fcy).

HEK293T cells (1.5×10⁶/well) were seeded in 6-well plates coated with 0.1 w/v % gelatin. DMEM containing 10% FBS and 1% nonessential amino acids (NEAA) was used as the medium. The pLV-hEPAS1 plasmid (4 μg), packaging plasmid psPAX2 (Addgene, #12260) (2 μg) and envelope plasmid (pMD2.G) (Addgene, #12259) (2 μg) were transfected into HEK293T cells (2 wells) using 500 μL of Opti-MEM (Thermo Fisher Scientific) and polyethyleneimine (Polysciences inc.). After 20 to 24 hours, the entire medium was replaced with DMEM containing 10% FBS, 1% nonessential amino acids (NEAA), and 1% P/S, and after another 24 and 48 hours, the entire medium was replaced with fresh medium, and the culture supernatant was collected. The culture supernatant was filtered through a PVDF syringe filter (0.45 μm), and the Lenti-X Concentrator (Clontech) was added and centrifuged to obtain a virus pellet. The pellet was then suspended in the medium to be used, and the virus suspension was stored at −80° C.

Human iPS cells (201B7 strain) were seeded at a concentration of 6×10⁴ cells/well, using mTeSR1 supplemented with 10 μM of Y-27632 (day 0) on a 6-well plate coated with iMatrix. The next day, the medium was replaced with fresh mTeSR1 (2 mL/well), and 200 μL/well of virus suspension was added (day 1). Thereafter, the medium was replaced daily with fresh mTeSR1, and 4 days after adding the virus, the medium was replaced with mTeSR1 supplemented with 0.3 μg/mL of puromycin (day 5). Puromycin selection was performed by culturing cells in mTeSR1 supplemented with 0.3 μg/mL of puromycin for at least 10 days after adding the virus. The expression of EPAS1 in selected iPS cells (normally oxygenated) was analyzed using qPCR. In addition, the selected iPS cells were induced into glomus cell-like cells by the procedure described in (1-4) above, and the expression of EPAS1 in the cells on day 17 of induction (normally oxygenated conditions) was analyzed by qPCR. The qPCR was performed by the same procedure as described above (3-3).

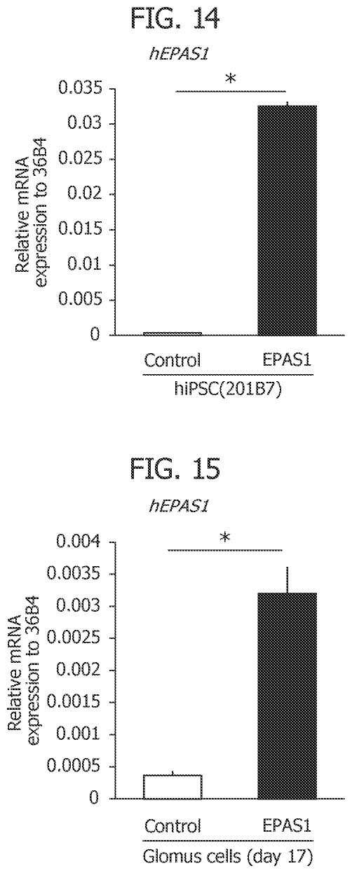

Results are shown in FIGS. 14 and 15. In the figures, “Control” indicates wild-type iPS cells. It was confirmed that EPAS1-introduced iPS cells strongly expressed EPAS1 (FIG. 14, *P<0.05, n=3, Student's t-test). Based on these results, iPS cells introduced with EPAS1 are hereinafter described as “EPAS1-expressing iPS cells.” Glomus cell-like cells derived from EPAS1-expressing iPS cells also strongly expressed EPAS1 (FIG. 15, *P<0.05, n=3, Student's t-test). Based on these results, glomus cell-like cells derived from EPAS1-expressing iPS cells are hereafter referred to as “EPAS1-overexpressing glomus cell-like cells.”

<5. Gene Expression in Glomus Cell-Like Cells Prepared from EPAS1-Expressing iPS Cells>

Expression of the glomus cell marker gene TH and glomus cell-associated factors KCNK3 and OR51E2 were analyzed using qPCR in EPAS1-expressing iPS cells (undifferentiated, day 3 of induction), autonomic neural progenitor cells induced from EPAS1-expressing iPS cells (day 13 of induction), and EPAS1-overexpressing glomus cell-like cells (day 17 of induction), qPCR was performed using the same procedure as described above (3-3).

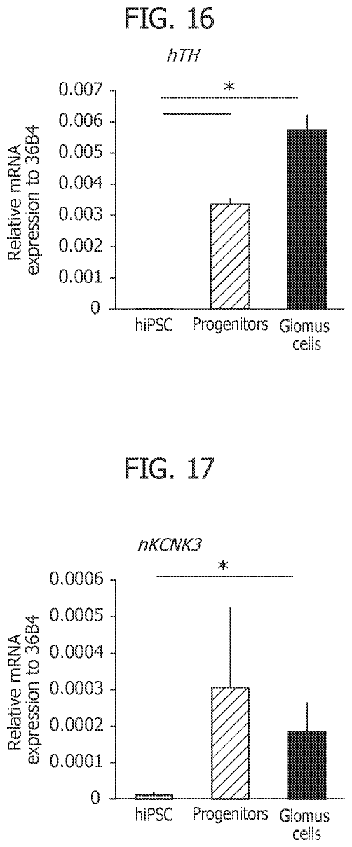

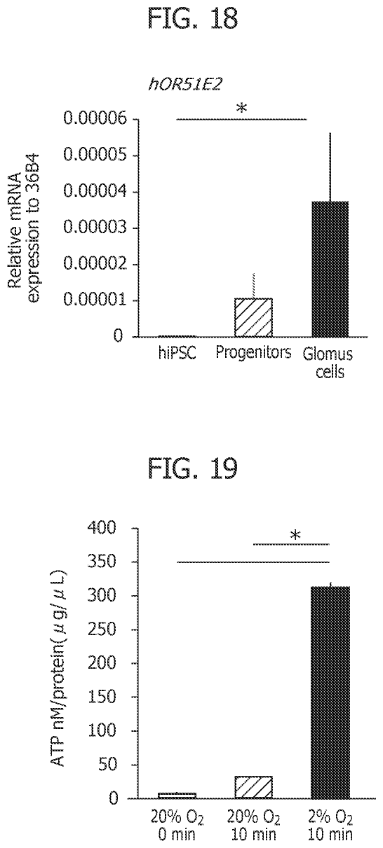

The results are shown in FIGS. 16 to 18. In the figures, “hiPSC” refers to EPAS1-expressing iPS cells, “progenitors” refers to autonomic neural progenitor cells induced from EPAS1-expressing iPS cells (day 13 of induction), and “glomus cells” refer to EPAS1-overexpressing glomus cell-like cells (day 17 of induction). It was confirmed that TH, KCNK3, and OR51E2 expression increased with differentiation (*P<0.05, n=3, Student's t-test).

<6. Hypoxic Response of Glomus Cell-Like Cells Prepared from EPAS1-Expressing iPS Cells>

### (6-1) ATP Production

ATP production by EPAS1-overexpressing glomus cell-like cells (day 17 of induction) in response to hypoxic stimulation was analyzed by the procedure described above (3-1). EPAS1-overexpressing glomus cell-like cells (day 17 of induction) under normoxic conditions (20% O₂) were used as controls.

The results are shown in FIG. 19 (*P<0.05, n=3, Student's t-test). EPAS1-overexpressing glomus cell-like cells showed high ATP production in response to hypoxic stimulation, and the amount of ATP production was 10-fold higher than that of glomus cell-like cells prepared from iPS cells without EPAS1 (FIG. 4). These results confirmed that EPAS1-overexpressing glomus cell-like cells are highly sensitive to hypoxic conditions and have high hypoxia responsiveness.

<7. Screening for Compounds that Regulate Glomus Cell Activity>

Eighty-seven commercial compounds listed in Table 2 below were screened for their ability to regulate glomus cell activity. Glomus cell-like cell spheres on days 16 to 19 of induction, prepared according to the procedure described in 1 above, were washed twice with KRB, and KRB containing the compound at 4 to 5 spheres/well (96-well plates) was added, and the ATP production amount was analyzed by the procedure described above (3-1). KRB containing DMSO (used as a solvent for the compound) was used as a control.

TABLE 2
Screened compounds

| No. | Compound | CAS No. | Distributor | Product No. | Lot No. | Category |
|---|---|---|---|---|---|---|
| 1 | Pifithrin-α (cyclic) | | Calbio Chem | 508-43391 | B60824 | p53 |
| 2 | PRIMA-1 | 5608-24-2 | ALEXIS | 270-310-M001 | 63520 | p53 activator |
| 3 | Finasteride | 98319-26-7 | LKT | F3354 | 239225221 | 5α-reductase |
| 4 | Aminoglutethimide | 125-84-8 | MPB | 153645 | 6145F | aromatase |
| 5 | Formestane | 566-48-3 | LKT | F5769 | 23921804 | aromatase |
| 6 | Mifepristone | 84371-65-3 | TOCRIS | 576-77361 | 1A/71494 | progesterone |
| | | | | | | receptor |
| 7 | TOFA | 54857-86-2 | Calbio Chem | 613450 | B58486 | acetyl-CoA |
| | | | | | | carboxylase (ACC) |
| 8 | Amastatin | 100938-10-1 | SIGMA | 1002018088 | SLBJ7839V | aminopeptidase A |
| 9 | Actinonin | 13434-13-4 | ALEXIS | 260-128-M005 | L06456 | aminopeptidase M |
| 10 | Oligomycin | 1404-19-9 | WAKO | 159-02181 | CEG1727 | F1-ATPase |
| 11 | Bafilomycin A1 | 88899-55-2 | KOM (Bio | BVT-0252- | B1810171 | V-ATPase |
| | | | Viotica) | M001 | | |
| 12 | HA 14-1 | 65673-63-4 | Calbio Chem | 371971 | E1013 | Bcl-2 |
| 13 | BH3I-1 | 300817-68-9 | ALEXIS | 430-122-M005 | L14467/a | Bcl-XL |
| 14 | LFM-A13 | 244240-24-2 | CAYMAN | 10010265 | 0435670-8 | Burton's tyrosine |
| | | | | | | kinase (BTK) |
| 15 | Terreic acid | 121-40-4 | TOCRIS | 557-75851 | 1A/64980 | Burton's tyrosine |
| | | | | | | kinase (BTK) |
| 16 | E-64d | 88321-09-9 | CAYMAN | 13533 | 0485057-19 | calpain |
| 17 | ALLN | 110044-82-1 | MPB | 598-00131 | 7083B | calpain, cathepsin |
| | | | | | | B, L |
| 18 | CA-074 | 134448-10-5 | PEPTIDE | 337-43221 | 540230 | cathepsin B |
| | | | INSTITUTE, Inc. | | | |
| 19 | Pepstatin A | 26305-03-3 | CAYMAN | 9000469 | 0512950-5 | cathepsin D |
| 20 | Z-GLF-CMK | | SIGMA | C9984 | 122K1381 | cathepsin G |
| 21 | RS 102895 | 300815-41-2 | TOCRIS | 2089 | 1A/69949 | CCR2 |
| 22 | SB 328437 | 247580-43-4 | Calbio Chem | 559406 | D00162186 | CCR3 |
| 23 | SB 225002 | 182498-32-4 | Calbio Chem | 559405 | D00148807 | CXCR2 |
| 24 | AMD3100 | 155148-31-5 | SIGMA | A5602 | 012M4618v | CXCR4 |
| | octahydrochloride | | | | | |
| 25 | NSC95397 | 93718-83-3 | TOCRIS | 1547 | 1A/67872 | Cdc25 |
| 26 | SC-αασ9 | 219905-91-6 | SIGMA | S2938 | 81K4610 | Cdc25A |
| 27 | Amiloride | 2016-88-8 | Calbio Chem | 535-79211 | B70676 | Na channel |
| 28 | Lidocaine | 73-78-9 | WAKO | 120-02691 | LTM0644 | Na channel |
| 29 | Monensin | 22373-78-0 | Calbio Chem | 530-80371 | B56050 | Na ionophore |
| 30 | Ouabain | 630-60-4 | ChromaDex, | ASB- | 19365- | Na/K ATPase |
| | | | Inc. | 00019365-010 | AY01 | |
| 31 | Sanguinarine | 5578-73-4 | ChromaDex, | ASB-00019050- | 19050-202 | Na/K/Mg ATPase |
| | | | Inc. | 010 | | |
| 32 | Glibenclamide | 10238-21-8 | WAKO | 078-03881 | DPR1692 | K channel |
| 33 | Dequalinium | 522-51-0 | SIGMA | D3768 | 094k1564 | K channel |
| 34 | Diazoxide | 364-98-7 | SIGMA | D9035 | 045K1513 | K channel opener |
| 35 | Valinomycin | 2001-95-8 | WAKO | 228-01121 | CEF2622 | K ionophore |
| 36 | Nigericin | 28643-80-3 | LKT | N3225 | 2597466 | K ionophore |
| 37 | Diltiazem | 33286-22-5 | WAKO | 047-20311 | EWM2717 | Ca channel |
| 38 | Nifedipine | 21829-25-4 | MPB | 591-08203 | 3970H | Ca channel |
| 39 | Verapamil | 152-11-4 | WAKO | 222-00781 | EWH6262 | Ca channel, MDR |
| 40 | PGP-4008 | 365565-02-2 | ALEXIS | 270-290-M002 | 9121219 | MDR |
| 41 | Fumitremorgin C | 118974-02-0 | SIGMA | F9054 | 096M4074V | BCRP |
| 42 | A23187 | 52665-69-7 | WAKO | 019-20111 | CEH0092 | Ca ionophore |
| 43 | Ionomycin | 56092-81-0 | CAYMAN | 10004974 | 0507600-21 | Ca ionophore |
| 44 | Thapsigargin | 67526-95-8 | CAYMAN | 10522 | 0507161-18 | Ca-ATPase |
| 45 | t- | 1948-33-0 | WAKO | 027-07212 | LTM0590 | Ca-ATPase |
| | Butylhydroquinone | | | | | |
| | (BHQ) | | | | | |
| 46 | N- | 91-40-7 | WAKO | 164-01331 | CEK1799 | Cl channel |
| | phenylanthranilic | | | | | |
| | acid | | | | | |
| 47 | DIDS | 53005-05-3 | MPB | 592-03711 | 2640F | Cl channel |
| 48 | SB 218078 | 135897-06-2 | TOCRIS | 2560 | 1A/201302 | Chk 1 |
| 49 | Debromohymenial | 75593-17-8 | CAYMAN | 14873 | 0468555-1 | Chk 1, 2 |
| | disine (DBH) | | | | | |
| 50 | Rotenone | 83-79-4 | MPB | 599-10811 | 1730J | mitochondrial |
| | | | | | | complex I |
| 51 | Antimycin A1 | 1397-94-0 | MPB | 591-01221 | 1210J | mitochondrial |
| | | | | | | complex III |
| 52 | Leptomycin B* | 87081-35-4 | Dr. Yoshida | | | CRM1 |
| | | | (RIKEN) | | | |
| 53 | R59022 | 93076-89-2 | CAYMAN | 16772 | 0493865-8 | DAG kinase |
| 54 | Dioctanoylglycol | 627-86-1 | TOCRIS | 538-43171 | 1*A/39820 | DAG kinase |
| 55 | RHC80267 | 83654-05-1 | MPB | 159011 | 3183J | DAG lipase |
| 56 | Xanthohumol | 6754-58-1 | ALEXIS | 572-72961 | 4251318 | DAG |
| | | | | | | acyltransferase |
| | | | | | | (DGAT) |
| 57 | C75 | 191282-48-1 | ALEXIS | 270-286-M001 | 7251301 | fatty acid synthase |
| | | | | | | (FAS) |
| 58 | Cerulenin | 17397-89-6 | WAKO | 031-18181 | CER1961 | FAS |
| 59 | Tunicamycin | 11089-65-9 | WAKO | 202-08241 | TLM1026 | glycosylation |
| 60 | Deoxynojirimycin | 73285-50-4 | Calbio Chem | 260684 | D00153833 | glucosidase I, II |
| 61 | Swainsonine | 72741-87-8 | WAKO | 198-10281 | TLL2915 | α-mannosidase |
| 62 | LY 83583 | 91300-60-6 | WAKO | 128-04691 | CEPO425 | guanylate cyclase |
| 63 | ODQ | 41443-28-1 | WAKO | 153-01981 | LAG1477 | guanylate cyclase |
| 64 | Anacardic acid | 16611-84-c | ALEXIS | 270-381-M005 | 10291201 | HAT |
| 65 | Chetomin | 1403-36-7 | SIGMA | C9623 | 027M4135V | HIF |
| 66 | Dimethyloxalylglycine | 89464-63-1 | CAYMAN | 71210 | 146121- | HIF-1α hydroxylase |
| | | | | | 151880 | |
| 67 | HR22C16 | 462630-41-7 | Enzo | ALX-270-373- | 11141704 | kinesin Eg5 |
| | | | | M001 | | |
| 68 | Monastrol | 254753-54-3 | Calbio Chem | 475879 | D00153347 | kinesin Eg5 |
| 69 | Nordihydroguaiaretic | 500-38-9 | MPB | 592-08451 | 7323H | lipoxygenase |
| | acid (NDGA) | | | | | |
| 70 | ETYA | 1191-85-1 | CAYMAN | 90120 | 166103-21 | 12,15-lipoxygenase |
| 71 | Baicalein | 491-67-8 | WAKO | 027-07751 | LTJ4210 | 12-lipoxygenase |
| 72 | Nutlin-3 | 548472-68-0 | SIGMA | 1002300280 | 105M4737V | Mdm2 |
| 73 | MDM2 inhibitor | | Calbio Chem | 444145 | B70049 | Mdm2 |
| 74 | Phenelzine | 156-51-4 | USP | 1517006 | G | monoamine oxidase |
| 75 | Deprenyl | 4528-51-2 | MPB | 151469 | R22027 | monoamine oxdase B |
| 76 | Decylubiquinone | 55486-00-5 | MPB | 195041 | L13043 | mitochondrial |
| | | | | | | permeability transition |
| | | | | | | pore (MPTP) |
| 77 | Ro 5-4864 | 14439-61-3 | SIGMA | C5174 | 014M4087V | MPTP |
| 78 | Lonidamine | 50264-69-2 | SIGMA | L4900 | 095K4022 | MPTP opener |
| 79 | ML-7 | 110448-33-4 | Calbio Chem | 475880 | 3015521 | myosin light chain |
| | | | | | | kinase |
| 80 | Benzylguanine | 19916-73-5 | ALEXIS | 480-019-M010 | L07236 | O6-methylguanine- |
| | | | | | | DNA |
| | | | | | | methyltransferase |
| | | | | | | (MGMT) |
| 81 | DFMO | 68278-23-9 | Calbio Chem | 507-28081 | B65802 | ornithine |
| | | | | | | decarboxylase (ODC) |
| 82 | KT 5823 | 126643-37-6 | FCS | 10-2083 | S101185 | PKG |
| 83 | Rp-8-CPT-cGMPS | 153660-04-9 | Enzo | BML-CN-206- | 8261666 | PKG |
| | | | | 0001 | | |
| 84 | MK 886 | 118427-55-7 | WAKO | 130-13001 | 0410322-135 | PPAR-α |
| 85 | Clofibrate | 637-07-0 | WAKO | 039-10603 | EWN0811 | PPAR-α activator |
| 86 | BADGE | 1675-54-3 | TOCRIS | 1326 | 2AI65024 | PPAR-γ |
| 87 | Troglitazone | | Calbio Chem | 571-71691 | B69840 | PPAR-γ activator |

The results showed that 19 compounds increased ATP production by 1.5-fold or more, and 19 compounds decreased ATP production by 0.5-fold or less compared to the control (data not shown). In addition, nifedipine was included among the compounds that decreased ATP production. Nifedipine has already been reported to suppress the hypoxic response of rat glomus cells (Buttigieg J. and Nurse CA., Biochem. Biophys. Res. Commun., 2004; 322(1): 82-7). Therefore, the above results supported the idea that this screening method can accurately screen compounds that regulate glomus cell activity.

1. A method for producing glomus cell-like cells, comprising
(a) a step of inducing pluripotent stem cells to differentiate into autonomic neural progenitor cells, and
(b) a step of culturing autonomic neural progenitor cells obtained by the step (a) in the presence of an FGF signaling pathway activator, an EGF signaling pathway activator, and an IGF-1 signaling pathway activator.

2. The method according to claim 1 further comprising (c) a step of introducing an exogenous nucleic acid encoding EPAS1 into the pluripotent stem cells, before the step (a).

3. The method according to claim 1, wherein the FGF signaling pathway activator is bFGF.

4. The method according to claim 1, wherein the EGF signaling pathway activator is EGF.

5. The method according to claim 1, wherein the IGF-1 signaling pathway activator is IGF-1.

6. The method according to claim 1, wherein the pluripotent stem cells are human-derived.

7. Glomus cell-like cells obtained by the method according to claim 1.

8. A glomus cell-like cell comprising an exogenous nucleic acid encoding EPAS1 and expressing TH, KCNK3, and OR51E2.

9. The glomus cell-like cell according to claim 7, which increases the production of ATP, or dopamine or a metabolite thereof, under hypoxic conditions.

10. A method of screening for a compound that regulates the activity of glomus cells, comprising:
(1) a step of contacting a candidate compound with the glomus cell-like cell according to claim 7, and
(2) a step of measuring ATP released from the glomus cell-like cells.


SEQUENCE LISTING (10 sequences)

SEQ ID NO: 1 - DNA, 18 bp, synthetic construct
  misc_feature 1..18: note = 36B4-F
  source 1..18: mol_type = other DNA
agatgcagcagatccgca

SEQ ID NO: 2 - DNA, 19 bp, synthetic construct
  misc_feature 1..19: note = 36B4-R
  source 1..19: mol_type = other DNA
gttcttgcccatcagcacc

SEQ ID NO: 3 - DNA, 18 bp, synthetic construct
  misc_feature 1..18: note = hTH-F
  source 1..18: mol_type = other DNA
gcgcaggaagctgattgc

SEQ ID NO: 4 - DNA, 20 bp, synthetic construct
  misc_feature 1..20: note = hTH-R
  source 1..20: mol_type = other DNA
caatctcctcggcggtgtac

SEQ ID NO: 5 - DNA, 21 bp, synthetic construct
  misc_feature 1..21: note = hKCNK3-F
  source 1..21: mol_type = other DNA
ctacgagcactggaccttctt

SEQ ID NO: 6 - DNA, 23 bp, synthetic construct
  misc_feature 1..23: note = hKCNK3-R
  source 1..23: mol_type = other DNA
cgtaaggatgtagacgaagctga

SEQ ID NO: 7 - DNA, 20 bp, synthetic construct
  misc_feature 1..20: note = hOR51E2-F
  source 1..20: mol_type = other DNA
ccgaactgctgtatgggctc

SEQ ID NO: 8 - DNA, 21 bp, synthetic construct
  misc_feature 1..21: note = hOR51E2-R
  source 1..21: mol_type = other DNA
gcatgtgtgaagttgcaggaa

SEQ ID NO: 9 - DNA, 20 bp, synthetic construct
  misc_feature 1..20: note = hEPAS1-F
  source 1..20: mol_type = other DNA
gtctgcaaagggttttgggg

SEQ ID NO: 10 - DNA, 19 bp, synthetic construct
  misc_feature 1..19: note = hEPAS1-R
  source 1..19: mol_type = other DNA
tgtgaggtgctgccaccag